(19) United States
(12) Patent Application Publication
(10) Pub. No.: US 2026/0191758 A1
(43) Pub. Date: Jul. 9, 2026

(54) SOLID COSMETIC FOR LIPS

(71) Applicant: SHISEIDO COMPANY, LTD., Chuo-ku, Tokyo (JP)

(72) Inventors: Mizuki YAMAMOTO, Tokyo (JP); Noriko TOMITA, Tokyo (JP)

(73) Assignee: SHISEIDO COMPANY, LTD., Chuo-ku, Tokyo (JP)

(21) Appl. No.: 19/133,030

(22) PCT Filed: Dec. 14, 2023
(86) PCT No.: PCT/JP2023/044908

§ 371 Date: May 27, 2025

(30) Foreign Application Priority Data
Dec. 28, 2022 (JP) 2022-212074

(51) Int. Cl. A61K 8/85; A61K 8/19; A61K 8/31; A61K 8/58; A61K 8/92; A61Q 1/06

(52) U.S. Cl. CPC A61K 8/85; A61K 8/19; A61K 8/31; A61K 8/585; A61K 8/922; A61Q 1/06; A61K 2800/43; A61K 2800/612; A61K 2800/651

(57) ABSTRACT

The present invention addresses the problem of providing a solid cosmetic for lips in which transfer resistance (secondary adhesion prevention effect) is further improved. The present invention pertains to a solid cosmetic for lips, characterized by containing: (A) a color material surface-treated with a treatment agent containing an amino acid or a metal soap; (B) 15-50 mass % of at least one substance selected from (b1) copolymers containing a dimer acid ester, and (b2) dimer acid esters; (C) 20-70 mass % of (c1) a non-volatile hydrocarbon oil that separates when mixed with (B) at 25° C. or (c2) a phenyl-modified silicone that separates when mixed with (B) at 25° C.; and (D) wax.

### TECHNICAL FIELD

The present invention relates to a solid cosmetic for lips (a solid lip cosmetic). More specifically, the present invention relates to a solid cosmetic for lips with exceptional transfer resistance (“secondary adhesion-less effect” or “secondary adhesion resistance effect”) in particular.

### BACKGROUND ART

When makeup cosmetics such as lipstick come into contact with dishes or clothing, the color may transfer (secondary adhesion) to the contact objects. Secondary adhesion not only makes the color of the makeup adhere on the contact object, but can also be a cause of messy makeup. Lipstick, in particular, is prone to secondary adhesion as the lips frequently come into contact with dishes. Therefore, cosmetics have been developed with so-called secondary adhesion-less effect, in which color transfer due to secondary adhesion is suppressed (see, for example, Patent Document 1).

The solid cosmetic for lips described in Patent Document 1 comprises (a) 35 to 50 mass % of hydrogenated polyisobutene, (b) 20 to 60 mass % of one or more methyl phenyl silicones, which separate when mixed with the component (a) at 25° C., (c) 1 to 20 mass % of neopentyl glycol dicaprate as a compatibility adjuster to adjust the compatibility of the component (a) and the component (b), and (d) 3 to 12 mass % of a wax (claim 1). The solid cosmetic for lips may further comprise (e) a color material (claim 2).

The solid cosmetic for lips described in Patent Document 1 is prepared in a uniform single-phase state, but when the cosmetic is applied to the lips, shear forces and/or pressure during application and pressing lips together cause the (a) hydrogenated polyisobutene (adhesion oil) and (b) methyl phenyl silicone (bleed oil or coating oil) to separate, which results in the adhesion oil adhering to the skin (lips) and the coating oil covering the adhesion oil. Since the color material blended in the cosmetic is mainly distributed in the adhesion oil, secondary adhesion (color transfer) of the color material is suppressed by covering it with a colorless, transparent coating oil.

The solid cosmetic for lips described in Patent Documents 2 and 3 are said to have excellent glossy appearance in addition to secondary adhesion-less effect, without needing to contain (c) the compatibility adjuster (also referred to as “compatibilizing agent” or “binding oil”) of Patent Document 1 by using a copolymer containing a dimer acid ester and/or a dimer acid ester as the adhesion oil.

However, since the adhesion oil and the coating oil are in contact with each other, some of the color material will inevitably transfer from the adhesion oil to the coating oil. Therefore, it was not possible to completely prevent the color material that had transferred to the coating oil from being transferred to the contact object (secondary adhesion).

### CITATION LIST

### Patent Document

Patent Document 1: JP-B 6242958
Patent Document 2: JP-A 2022-115395
Patent Document 3: JP-A 2022-115396

### SUMMARY OF INVENTION

### Technical Problem

In view of the current state of the arts described above, the present invention aims to provide a solid cosmetic for lips with further improved transfer resistance (secondary adhesion-less effect).

### Solution to Problem

As a result of extensive studies to solve the aforementioned problem, the present inventors have found that the transfer of the color material from the adhesion oil to the coating oil can be suppressed and the transfer resistance (secondary adhesion-less effect) can be further enhanced by treating the surface of the color material to be blended in the cosmetic with a specific surface-treating agent, and thereby completed the present invention.

That is, the present invention provides a solid cosmetic for lips, comprising:
(A) a color material surface-treated with a treatment agent comprising an amino acid or a metallic soap;
(B) 15 to 50 mass % of at least one selected from (b1) a copolymer comprising a dimer acid ester and (b2) a dimer acid ester;
(C) 20 to 70 mass % of (c1) a non-volatile hydrocarbon oil that separates when mixed with (B) at 25° C., or (c2) a phenyl-modified silicone that separates when mixed with (B) at 25° C.; and
(D) a wax.

### Advantageous Effects of Invention

The solid cosmetic for lips of the present invention has excellent adhesion and glossy appearance by being formulated with a copolymer containing a dimer acid ester and/or a dimer acid ester as the adhesion oil. In addition to this, being formulated with a color material that has undergone a specific surface treatment allows to suppress the transfer of the color material to the coating oil, resulting in a cosmetic with exceptional secondary adhesion-less effect (transfer resistance).

### DESCRIPTION OF EMBODIMENTS

The solid cosmetic for lips of the present invention (hereinafter, also simply referred to as “cosmetic”) contains, as essential components, (A) a color material surface-treated with an amino acid or a metallic soap; (B) at least one selected from (b1) a copolymer containing a dimer acid ester and (b2) a dimer acid ester; (C) (c1) a non-volatile hydrocarbon oil or (c2) a phenyl-modified silicone that separates when mixed with (B) at 25° C.; and (D) a wax.

### (A) Color Material

The “color material” (also referred to as “component A”) blended in the cosmetic of the present invention includes color materials that have been surface-treated with a treatment agent containing an amino acid or a metallic soap.

A treatment agent containing an amino acid is a surface-treating agent containing at least one amino acid or derivative thereof.

The “amino acid” is not particularly limited, but is preferably selected from aspartic acid, glutamic acid, lysine, proline, and sarcosine, of which glutamic acid is particularly preferred.

The “derivative of an amino acid” is preferably an N-acyl amino acid or a salt thereof. The “N-acyl amino acid or salt thereof” is a compound in which an acyl group, preferably a saturated fatty acid having 12 to 20 carbon atoms, is condensed on an amino group of an amino acid, or a salt thereof. Examples of the “acyl group” include a stearoyl group, a lauroyl group, a palmitoyl group, and a cocoyl group. The “salt” can be selected from alkali metal salts such as sodium and potassium, and alkaline earth metal salts, but is preferably a sodium salt. Specific examples of the N-acyl amino acid salt include disodium N-stearoyl glutamate, sodium N-lauroyl glutamate, sodium lauroyl aspartate, sodium palmitoyl sarcosinate, and magnesium palmitoyl glutamate, but are not limited thereto.

The treatment agent containing an amino acid may contain at least one other treatment agent in addition to the “amino acid or derivative thereof”. The “other treatment agent” is preferably selected from “metal hydroxides,” “lipids,” and “esters.” Examples of the “metal hydroxides” include aluminum hydroxide, and examples of the “lipids” include higher fatty acids such as “palmitic acid.” Examples of the “esters” include esters of monovalent or divalent fatty acids having 8 to 12 carbon atoms and saturated aliphatic alcohols having 12 to 20 carbon atoms. The alkyl chain of the fatty acids and aliphatic alcohols may be linear or branched. As the “ester,” isostearyl sebacate is particularly preferably used.

Examples of the “treatment agent containing an amino acid” include a surface-treating agent containing disodium N-stearoyl glutamate and aluminum hydroxide, a surface-treating agent containing disodium N-stearoyl glutamate, isostearyl sebacate, and aluminum hydroxide, a surface-treating agent containing sodium dilauramidoglutamide lysine and magnesium chloride, and a surface-treating agent containing palmitoyl proline, sodium palmitoyl sarcosinate, magnesium palmitoyl glutamate, and palmitic acid. Of these, it is preferable to use a color material that has been surface-treated with a treatment agent containing disodium N-stearoyl glutamate and aluminum hydroxide or a treatment agent containing disodium N-stearoyl glutamate, isostearyl sebacate, and aluminum hydroxide.

The treatment agent containing a metallic soap is a surface-treating agent containing at least one metallic soap.

The “metallic soap” is a salt of a saturated or unsaturated higher fatty acid with a metal (but other than alkali metals). Although not particularly limited, examples of the higher fatty acids constituting the metallic soap include saturated and/or unsaturated higher fatty acids having 8 to 24, particularly 12 to 18 carbon atoms, such as stearic acid, isostearic acid, myristic acid, and lauric acid. A salt of aluminum, calcium, magnesium, or zinc is preferred as the metal constituting the metallic soap.

Specific examples of the metallic soap preferably used as a surface-treating agent of the color material (component A) in the present invention include magnesium stearate, calcium stearate, zinc stearate, calcium laurate, zinc laurate, calcium ricinoleate, zinc ricinoleate, zinc octylate, and aluminum myristate.

The surface treatment with a metallic soap can be performed by a method of surface-treating with a preformed metallic soap. For example, the surface treatment can be performed by dissolving a metallic soap in a volatile solvent such as isoparaffin or isopropyl alcohol, mixing it with a base powder (color material), and then volatilizing the volatile solvent. The surface treatment can also be performed by simply mixing a base powder (color material) with a metallic soap. Alternatively, the surface treatment may be performed by a method of composite treatment with a higher fatty acid (e.g., stearic acid) constituting a metallic soap, and a hydroxide of a metal (e.g., aluminum). The method of surface treatment used can be a wet method using a solvent, a gas phase method, a mechanochemical method, or the like, and is not particularly limited.

The base material for the “color material” (component A) is not particularly limited as long as it is a powdered color material. Examples thereof include inorganic white pigments (titanium dioxide and zinc oxide), inorganic red pigments (iron oxide (bengala) and iron titanate), inorganic brown pigments (gamma-ferric oxide), inorganic yellow pigments (yellow iron oxide and yellow ocher), inorganic black pigments (black iron oxide, carbon black, and lower titanium oxide), inorganic purple pigments (mango violet and cobalt violet), inorganic green pigments (chromium oxide, chromium hydroxide, and cobalt titanate), inorganic blue pigments (ultramarine and iron blue), pearl pigments (titanium oxide-coated mica, titanium oxide-coated bismuth oxychloride, titanium oxide-coated talc, colored titanium oxide-coated mica, bismuth oxychloride, and fish scale guanine), metal powder pigments (aluminum powder and copper powder), organic pigments (Red No. 202, Red No. 205, Red No. 220, Red No. 228, Red No. 405, Orange No. 203, Orange No. 204, Yellow No. 205, Yellow No. 40, and Blue No. 404), zirconium, barium and aluminum lake pigments (Red No. 3, Red No. 104, Red No. 227, Red No. 401, Orange No. 205, Yellow No. 4, Yellow No. 20, Green No. 3, and Blue No. 1), natural pigments (chlorophyll, carotenoids (β-carotene), carthamin, cochineal, chalcone, curcumin, betanin, flavonols, flavones, anthocyanidins, anthraquinone, and naphthoquinones), and functional pigments (boron nitride, photochromic pigments, synthetic fluorphlogopite, iron-containing synthetic fluorphlogopite, and hybrid fine powders).

The color material (component A) of the cosmetic of the present invention may consist of one or more color materials surface-treated with a treatment agent containing an amino acid, or one or more color materials surface-treated with a treatment agent containing a metallic soap. Alternatively, a color material surface-treated with a treatment agent containing an amino acid may be blended in combination with a color material surface-treated with a treatment agent containing a metallic soap. A color material surface-treated with a treatment agent free of amino acids and metallic soaps and an untreated color material may also be further contained.

The content of the color material (component A) in the cosmetic of the present invention is preferably 0.1 to 15 mass %, more preferably 1 to 12 mass %, and even more preferably 4 to 10 mass % with respect to the total amount of the cosmetic. The color material surface-treated with a treatment agent containing an amino acid or a metallic soap preferably accounts for 50 mass % (mass ratio) or more, more preferably 60 mass % or more, and even more preferably 70 mass % or more of the total amount of the color material (component A) blended in the cosmetic.

The “component B” (also referred to as “adhesion oil”) of the cosmetic of the present invention is at least one selected from (b1) a copolymer containing a dimer acid ester and (b2) a dimer acid ester.

### (b1) Copolymer Containing Dimer Acid Ester

The “copolymer containing a dimer acid ester” (hereinafter, also referred to as “component b1”) is a high-viscosity, non-volatile ester oil. It is preferable to select a copolymer containing a dimer acid ester (component b1) that is compatible with the (c1) non-volatile hydrocarbon oil described below at 90° C. and separates at 25° C., and that is compatible with the (c2) phenyl-modified silicone described below at 100° C. and separates at 25° C.

Examples of the copolymer containing a dimer acid ester (component b1) include polyglyceryl-2 isostearate/dimer dilinoleate copolymer, commercially available as HAILUCENT ISDA (manufactured by Kokyu Alcohol Kogyo).

### (b2) Dimer Acid Ester

The dimer acid ester (hereinafter, also referred to as “component b2”) is an ester of a liquid fatty acid which has as a main component, a dibasic acid, C36 dicarboxylic acid, produced by dimerization of a C18 unsaturated fatty acid derived from a vegetable oil or fat, and contains monobasic and tribasic acids.

Examples of the dimer acid ester (component b2) include hydrogenated castor oil dimer dilinoleate and dimer dilinoleyl dimer dilinoleate. Examples of commercially available products include RISOCAST DA-L (manufactured by Kokyu Alcohol Kogyo) and LUSPLAN DD-DA (manufactured by Nippon Fine Chemical).

The “component B” (adhesion oil) in the cosmetic of the present invention may contain either (b1) or (b2) alone or may contain both (b1) and (b2).

In the present invention, it is preferable to contain (b1) a copolymer containing a dimer acid ester, and that the content of the (b1) copolymer containing a dimer acid ester be 50 mass % or more of the total amount of the “component B” (adhesion oil).

The content of the “component B” (total content of the component b1 and component b2) in the cosmetic of the present invention is 15 to 50 mass %, and from the viewpoint of adhesion to the lips and durability of the cosmetic effect, preferably 20 to 40 mass %, and even more preferably 25 to 35 mass % with respect to the total amount of the cosmetic.

The “component C” (also referred to as “coating oil”) of the cosmetic of the present invention is (c1) a non-volatile hydrocarbon oil that separates when mixed with the “component B” at 25° C., or (c2) a phenyl-modified silicone that separates when mixed with the “component B” at 25° C.

(c1) Non-Volatile Hydrocarbon Oil that Separates when Mixed with “Component B” at 25° C.

The non-volatile hydrocarbon oil that separates when mixed with the “component B” at 25° C. (hereinafter, also referred to as “non-volatile hydrocarbon oil” or “component c1”) is a non-volatile hydrocarbon oil liquid at normal temperature that can be blended in a cosmetic.

The “component c1” of the present invention separates when mixed with the (B) copolymer containing a dimer acid ester and dimer acid ester at 25° C. and is compatible at 90° C.

The terms “separate” and “compatible” in the present specification are defined as follows.

“Separate”: the “component B” and the “component c1” were defined as “separating” if they separate into two layers with a uniform (visible) boundary when they were heated to 90° C. at (B):(c1)=1:1 (mass ratio), mixed by stirring, then left to stand, and the mixture had reached 25° C., and they were defined as “not separating” if their mixture was transparent with no boundary.

“Compatible”: the “component B” and the “component c1” were defined as “compatible” when they were heated to 90° C. at (B):(c1)=1:1 (mass ratio), mixed by stirring, and the mixture was transparent with no visible boundary, i.e., “not separating.”

The non-volatile hydrocarbon oil (component c1) used has a viscosity of 100 mPa-s or less, preferably 50 mPa-s. If the viscosity exceeds 100 mPa-s, problems such as inability to mold and aggregation of the color material may occur. The viscosity in the present invention is the value measured with a B-type viscometer TVB-10M (manufactured by TOKI SANGYO CO., LTD.) at 25° C., rotor name M1, and 30 rpm.

Specific examples of the non-volatile hydrocarbon oil (component c1) preferably include squalane, hydrogenated polyisobutene, hydrogenated polydecene, liquid paraffin, light isoparaffin, and heavy liquid paraffin. Examples of commercially available products include NOMCORT HP100 (manufactured by The Nisshin OilliO Group) and Squalane (manufactured by Kuraray).

(c2) Phenyl-Modified Silicone that Separates when Mixed with “Component B” at 25° C.

The phenyl-modified silicone that separates when mixed with the “component B” at 25° C. (hereinafter, also referred to as “phenyl-modified silicone” or “component c2”) is a phenyl-modified silicone oil (liquid at normal temperature (25° C.)) that can be blended in a cosmetic. The phenyl-modified silicone (component c2) in the present invention is compatible when mixed with the above “component B” at 100° C. and separates at 25° C. The definitions of “separate” and “compatible” are as described above, except that in the case of “compatible,” the measurement is at 100° C. instead of 90° C.

The phenyl-modified silicone (component c2) in the present invention preferably has a viscosity of 300 mPa-s or less. If the viscosity exceeds 300 mPa-s, it may no longer be compatible with the adhesion oil at elevated temperatures, and the (D) wax described below may not dissolve uniformly, making molding impossible.

Specific examples of the phenyl-modified silicone (component c2) include trimethyl pentaphenyl trisiloxane, diphenyl dimethicone, diphenylsiloxy phenyl trimethicone, and phenyl trimethicone. Of these, trimethyl pentaphenyl trisiloxane and diphenylsiloxy phenyl trimethicone are preferred.

In the cosmetic of the present invention, it is preferable to use either the (c1) non-volatile hydrocarbon oil or (c2) phenyl-modified silicone as the “component C” (coating oil). When both the (c1) non-volatile hydrocarbon oil and (c2) phenyl-modified silicone are used, the content of the (c2) phenyl-modified silicone is preferably less than 5 mass % of the total amount of the cosmetic from the viewpoint of formulation stability.

The content of the “component C” (coating oil) in the cosmetic of the present invention is 20 to 70 mass %, preferably 30 to 55 mass %, and even more preferably 35 to 50 mass % with respect to the total amount of the cosmetic. If the content of the “component C” is less than 20 mass %, there will be less separation during application, and if it exceeds 70 mass %, the content of the other components becomes too small. In either case, a secondary adhesion-less effect is less likely to be obtained.

When a non-volatile hydrocarbon oil (component c1) is used as the “component C” (coating oil), the blending ratio of the “component B” to the “component c1” (B:c1) is preferably 1:0.5 to 1:4, more preferably 1:0.8 to 1:3.5 and even more preferably 1:1 to 1:2. Within this range, the “component B” and the “component c1” are appropriately compatible at 90° C. and separate at 25° C.

When a phenyl-modified silicone (component c2) is used as the “component C” (coating oil), the blending ratio of the “component B” to the “component c2” (B:c2) is preferably 1:0.5 to 1:4, and even more preferably 1:0.8 to 1:3.5. Within this range, the “component B” and the “component c2” are appropriately compatible at 100° C. and separate at 25° C.

### (D) Wax

The wax (hereinafter, also referred to as “component D”) blended in the cosmetic of the present invention is not particularly limited as long as it is commonly blended in a cosmetic. The wax (component D) in the present invention is preferably compatible with the “component B” and the “component C” at elevated temperatures (90° C. to 100° C.) and separates at normal temperature (25° C.).

Examples of the wax used in the present invention include carnauba wax, candelilla wax, polyethylene wax, sunflower seed wax (sunflower wax), beeswax, ceresin, microcrystalline wax, solid paraffin, Japan wax, and cera flava.

The content of the wax (component D) is preferably 5 to 15 mass %, and even more preferably 7 to 12 mass % with respect to the total amount of the cosmetic. If the content of the wax (component D) is 5 mass % or less, it may be less likely to solidify, and if it exceeds 15 mass %, the cosmetic may spread less well and lose its gloss.

In addition to the above components A to D, other optional components commonly used in solid cosmetics can be blended into the solid cosmetic for lips of the present invention within a range not impairing the effects of the present invention. Examples of the other optional components include, but are not limited to, oils (other than the components B to D), powders (other than the component A), polymer compounds, moisturizers, fragrances, antioxidants, preservatives, and beauty ingredients.

The solid cosmetic for lips of the present invention can be produced without being formulated with a “compatibilizing agent” (or “binding oil”) since it uses a dimer acid ester as the adhesion oil. However, a compatibilizing agent may be blended as long as it does not impair the effects of the present invention. Specific examples of the compatibilizing agent include diisostearyl malate, neopentyl glycol dicaprate, triethylhexanoin, pentaerythrityl tetraethylhexanoate, and caprylic/capric triglyceride. When a compatibilizing agent is blended, the content is preferably 20 mass % or less of the total amount of the cosmetic.

Powders (other than the component A) include spherical powders and plate-like powders.

Examples of spherical powders include spherical resin powders such as polymethyl methacrylate, organopolysiloxane elastomer, polystyrene, polyamide resin (nylon), polyethylene, copolymer of styrene and acrylic acid, benzoguanamine resin, polytetrafluoroethylene, and silicone resin.

Examples of plate powders include inorganic powders such as mica, synthetic mica, talc, sericite, aluminum oxide, magnesium oxide, zirconium oxide, magnesium carbonate, calcium carbonate, calcium sulfate, chromium oxide, chromium hydroxide, aluminum silicate, magnesium silicate, magnesium aluminum silicate, kaolin, silicon carbide, barium sulfate, bentonite, smectite, and boron nitride, organic powders such as N-acyllysine, and hybrid powders such as fine titanium oxide-coated titanated mica, fine zinc oxide-coated titanated mica, and barium sulfate-coated titanated mica.

The content of the powder (other than the component A) in the cosmetic of the present invention is preferably 30 mass % or less, and even more preferably 20 mass % or less with respect to the total amount of the cosmetic.

The cosmetic of the present invention is produced using a method widely used for oil-based solid cosmetics. For example, the cosmetic can be produced by heating each formulation component as necessary, mixing by stirring, filling into a mold, and cooling slowly.

The cosmetic of the present invention is suitable to be provided as a makeup cosmetic, particularly a solid cosmetic for lips with exceptional transfer resistance (secondary adhesion-less effect). Specifically, it is preferably provided in the form of a lipstick, lip gloss, lip base for priming, lipstick top coat, and lip balm.

### EXAMPLES

Hereinafter, the present invention will be described in further detail with reference to Examples, but the present invention is not limited to these Examples. Unless otherwise specified, the content is indicated in mass %.

Solid cosmetics for lips were prepared according to a conventional method, using the formulations listed in Tables 1 to 3 below. Tables 1 and 2 show formulations using a non-volatile hydrocarbon oil as the coating oil, and Table 3 shows formulations using a phenyl-modified silicone as the coating oil.

The characteristics of the cosmetic of each example in Tables 1 to 3 were measured and evaluated by the following methods.

### (1) Moldability

The moldability of the cosmetic of each example when filled into a mold was evaluated by professional panelists.
A: Good molding was achieved
B: Molding was achieved, but hardness was insufficient
C: Did not harden and could not be molded

### (2) Aggregation of Color Material

The cosmetic of each example was evaluated by visual observation by professional panelists.
A: No aggregation of the color material was observed
B: Partial aggregation of the color material was observed
C: The color material was aggregated

### (3) Appearance

The cosmetic of each example was evaluated by visual observation by professional panelists.
A: Uniform and transparent appearance
B: Some opaque portions were observed
C: Uneven appearance

### (4) Secondary Adhesion-Less Effect

Professional panelists actually used the cosmetics of each example and evaluated by visual observation whether (and to what degree) there was color transfer to contact objects.
S: Secondary adhesion was perfectly inhibited
A: Almost no secondary adhesion was observed
B: Slight secondary adhesion was observed
C: A lot of secondary adhesion was observed
-: Not measurable

TABLE 1

| | | | | Comparative |
|---|---|---|---|---|
| | Example 1 | Example 2 | Example 3 | Example 1 |
| Polyethylene wax/microcrystalline wax | 10 | 10 | 10 | 10 |
| Polyglyceryl-2 isostearate/dimer dilinoleate | 30 | 30 | 30 | 30 |
| copolymer | | | | |
| Squalane | 55 | 55 | 55 | 55 |
| Amino acid-treated iron oxide (*1) | 5 | — | — | — |
| Amino acid-treated iron oxide (*2) | — | 5 | — | — |
| Metallic soap-treated iron oxide (*3) | — | | 5 | — |
| Silicone-treated iron oxide | — | — | — | 5 |
| Red No. 202 | — | — | — | — |
| Total | 100 | 100 | 100 | 100 |
| Moldability | A | A | A | A |
| Aggregation of color material | A | A | A | A |
| Appearance | A | A | A | A |
| Secondary adhesion-less effect | S | S | S | A |

(*1) Iron oxide surface-treated with a treatment agent containing disodium N-stearoyl glutamate and aluminum hydroxide
(*2) Iron oxide surface-treated with a treatment agent containing disodium N-stearoyl glutamate, isostearyl sebacate, and aluminum hydroxide
(*3) Iron oxide surface-treated with magnesium stearate

TABLE 2

| | Example 4 | Example 5 | Example 6 | Example 7 | Example 8 |
|---|---|---|---|---|---|
| Polyethylene wax/microcrystalline wax | 10 | 10 | 10 | 10 | — |
| Sunflower seed wax | — | — | — | — | 10 |
| Polyglyceryl-2 isostearate/dimer | 30 | 15 | 15 | 30 | 30 |
| dilinoleate copolymer | | | | | |
| Hydrogenated castor oil dimer acid | — | 15 | — | — | — |
| Dimer dilinoleyl dimer dilinoleate | — | | 15 | — | — |
| Squalane | 55 | 55 | 55 | — | 55 |
| Hydrogenated polyisobutene | — | — | — | 55 | — |
| (viscosity: 28 mPa · s) | | | | | |
| Amino acid-treated iron oxide (*1) | 2 | 5 | 5 | 5 | 5 |
| Amino acid-treated iron oxide (*2) | 2 | — | — | — | — |
| Red No. 202 | 1 | — | — | — | — |
| Total | 100 | 100 | 100 | 100 | 100 |
| Moldability | A | A | A | A | A |
| Aggregation of color material | A | A | A | A | A |
| Appearance | A | A | A | A | A |
| Secondary adhesion-less effect | S | S | S | S | S |

(*1) Iron oxide surface-treated with a treatment agent containing disodium N-stearoyl glutamate and aluminum hydroxide
(*2) Iron oxide surface-treated with a treatment agent containing disodium N-stearoyl glutamate, isostearyl sebacate, and aluminum hydroxide

TABLE 3

| | | | | Comparative | Comparative |
|---|---|---|---|---|---|
| | Example 9 | Example 10 | Example 11 | Example 2 | Example 3 |
| Polyethylene wax/microcrystalline | 10 | 10 | 10 | 10 | 10 |
| wax | | | | | |
| Polyglyceryl-2 isostearate/dimer | 30 | 15 | 15 | 30 | — |
| dilinoleate copolymer | | | | | |
| Hydrogenated castor oil dimer | — | 15 | — | — | — |
| acid | | | | | |
| Dimer dilinoleyl dimer dilinoleate | — | — | 15 | — | — |
| Hydrogenated polyisobutene | — | — | — | — | 30 |
| (viscosity: 20,000 mPa · s) | | | | | |
| Trimethyl pentaphenyl trisiloxane | 55 | 55 | 55 | 55 | 55 |
| Amino acid-treated iron oxide (*1) | 5 | 5 | 5 | — | 5 |
| Silicone-treated iron oxide | — | — | — | 5 | — |
| Total | 100 | 100 | 100 | 100 | 100 |
| Moldability | A | A | A | A | C |
| Aggregation of color material | A | A | A | A | C |
| Appearance | A | A | A | A | C |
| Secondary adhesion-less | S | S | S | A | — |
| effect | | | | | |

(*1) Iron oxide surface-treated with a treatment agent containing disodium N-stearoyl glutamate and aluminum hydroxide

Next, to quantify the improvement in the secondary adhesion-less effect, the following measurements were performed on the cosmetics of “Example 1” and “Comparative Example 1” in Table 1 and “Example 9” and “Comparative Example 2” in Table 3.

2.5 mg of each of the aforementioned cosmetics was applied on an artificial leather with an area of 4 cm². The artificial leather was folded with the applied surface facing inwards, rubbed between fingers 5 times, and then left to stand for 5 minutes. The applied area was then tapped twice with a filter paper using a tapping tester, and the lightness (L value) of the filter paper on which the cosmetic adhered was measured with a spectrophotometric colorimeter CM-2600d (manufactured by Konica Minolta). Based on the L values obtained for the cosmetic of each example, Table 4 shows the difference in L values (ΔL) obtained by subtracting the L value of the cosmetic formulated with a silicone-treated color material (Comparative Examples) from the L value of the cosmetic formulated with an amino acid-treated color material (Examples).

The “L value” is a numerical value representing “lightness” in the Lab color space, and the higher the “L value,” the stronger the “whiteness,” i.e., the less cosmetic has been transferred onto the filter paper.

TABLE 4

| | ΔL |
|---|---|
| Example 1 | 9.23 |
| Comparative Example 1 | |
| Example 9 | 6.05 |
| Comparative Example 2 | |

As shown in Table 4, whether a “(c1) non-volatile hydrocarbon oil” or a “(c2) phenyl-modified silicone” was used as the coating oil, color transfer was found to be more suppressed in the Examples in which an amino acid-treated color material was blended as the (a) color material, than in the Comparative Examples in which a silicone-treated color material was blended, with a significantly large difference (ΔL). That is, it can be said that there is a marked difference between the “S” and “A” ratings in the evaluation results of Tables 1 to 3 above.

The test results shown in Tables 1 to 3 indicate that the use of an amino acid-treated or metallic soap-treated color material as the color material remarkably suppressed secondary adhesion compared to when a silicone-treated color material was used, even when the composition of the adhesion oil, coating oil, color material, or wax was changed.

When a high-viscosity hydrogenated polyisobutene was used as the adhesion oil, the color material aggregated, making molding impossible (Comparative Example 3).

1. A solid cosmetic for lips, comprising:
(A) a color material surface-treated with a treatment agent comprising an amino acid or a metallic soap;
(B) 15 to 50 mass % of at least one selected from (b1) a copolymer comprising a dimer acid ester and (b2) a dimer acid ester;
(C) 20 to 70 mass % of (c1) a non-volatile hydrocarbon oil that separates when mixed with (B) at 25° C., or (c2) a phenyl-modified silicone that separates when mixed with (B) at 25° C.; and
(D) a wax.

2. The cosmetic according to claim 1, wherein the (A) color material is a color material surface-treated with a treatment agent comprising an N-acyl amino acid.

3. The cosmetic according to claim 2, wherein the N-acyl amino acid is N-stearoyl glutamic acid.

4. The cosmetic according to claim 1, wherein the (A) color material is a color material surface-treated with a treatment agent comprising a metallic soap.

5. The cosmetic according to claim 4, wherein the metallic soap is at least one selected from the group consisting of magnesium stearate, calcium stearate, zinc stearate, calcium laurate, zinc laurate, calcium ricinoleate, zinc ricinoleate, zinc octylate, and aluminum myristate.

6. The cosmetic according to claim 1, wherein the (b1) copolymer comprising a dimer acid ester is a polyglyceryl-2 isostearate/dimer dilinoleate copolymer.

7. The cosmetic according to claim 1, wherein the (b2) dimer acid ester is selected from hydrogenated castor oil dimer dilinoleate and dimer dilinoleyl dimer dilinoleate.

8. The cosmetic according to claim 1, wherein the (B) comprises (b1) a copolymer comprising a dimer acid ester, and a content of the (b1) copolymer comprising a dimer acid ester is 50 mass % or more of the total amount of (B).

9. The cosmetic according to claim 1, wherein the (c1) non-volatile hydrocarbon oil is selected from squalane, hydrogenated polyisobutene, hydrogenated polydecene, liquid paraffin, light isoparaffin, and heavy liquid paraffin.

10. The cosmetic according to claim 1, wherein the (c2) phenyl-modified silicone is selected from trimethyl pentaphenyl trisiloxane, diphenyl dimethicone, and diphenylsiloxy phenyl trimethicone.
